(19) United States
(12) Patent Application Publication
(10) Pub. No.: US 2022/0031886 A1
(43) Pub. Date: Feb. 3, 2022

(54) COMBINATION GLOVE AND ANTISEPTIC DISPENSER

(71) Applicant: GIFTEDNESS AND CREATIVITY COMPANY, Safat (KW)

(72) Inventor: MESHARI S.M.J. AL-MUTAIRI, SAFAT (KW)

(21) Appl. No.: 17/315,242

(22) Filed: May 7, 2021

Related U.S. Application Data
(60) Provisional application No. 63/057,905, filed on Jul. 29, 2020.

(51) Int. Cl. A61L 2/18; A41D 19/00; A41D 31/10

(52) U.S. Cl. CPC A61L 2/18; A41D 19/0024; A61L 2202/26; A61L 2202/16; A41D 31/10

(57) ABSTRACT

The combination glove and antiseptic dispenser includes a glove adapted for receiving the hand of the user and having an inner surface and an outer surface. The glove is formed from a first fluid-impermeable material. A dispensing layer is formed from at least one sheet, which is made from a second fluid-impermeable material and has a plurality of pores formed therethrough. At least one absorbent layer is sandwiched between the outer surface of the glove and the dispensing layer, such that antiseptic liquid absorbed by the at least one absorbent layer may be dispensed through the plurality of pores of the at least one sheet. The at least one absorbent layer may be formed from any suitable type of absorbent material, such as those commonly associated with pre-moistened towelettes, wet wipes, baby wipes and the like.

### CROSS-REFERENCE TO RELATED APPLICATION

This application claims the benefit of U.S. Provisional Patent Application No. 63/057,905, filed on Jul. 29, 2020.

### BACKGROUND

### 1. Field

The disclosure of the present patent application relates to protective garments, and particularly to a combination glove and antiseptic dispenser.

### 2. Description of the Related Art

Medical gloves are disposable gloves used during medical examinations and procedures to help prevent cross-contamination between caregivers and patients. They are also used to handle materials which may have been infected with bacteria, viruses and the like. Medical gloves can be made from different types of polymers, including latex, nitrile rubber, polyvinyl chloride and neoprene. Although these materials are fluid impermeable and non-porous, no known material is truly 100% effective at preventing transfer of infectious contaminants. Although medical gloves are often used in combination with antiseptic fluids, particularly in sterile medical environments, the antiseptic fluids are kept in separate containers and must be retrieved, handled, operated, transported and stored. Thus, at the time of use, it may be inefficient or difficult to take advantage of the additional protection offered by a separately stored antiseptic. Thus, a combination glove and antiseptic dispenser solving the aforementioned problems is desired.

### SUMMARY

The combination glove and antiseptic dispenser is a glove, such as those used in medical and laboratory environments, for example, providing a fluid, microbial and viral impermeable barrier for the user's hand and, additionally, dispensing an antiseptic liquid. The combination glove and antiseptic dispenser includes a glove adapted for receiving the hand of the user and having an inner surface and an outer surface. The glove is formed from a first fluid-impermeable material.

A dispensing layer is formed from at least one sheet, which is made from a second fluid-impermeable material and has a plurality of pores formed therethrough. The first fluid-impermeable material may be the same as the second fluid-impermeable material. A non-limiting example of such a material is nitrile rubber.

At least one absorbent layer is sandwiched between the outer surface of the glove and the dispensing layer, such that antiseptic liquid absorbed by the at least one absorbent layer may be dispensed through the plurality of pores of the at least one sheet. It should be understood that the at least one absorbent layer may be formed from any suitable type of absorbent material, such as those commonly associated with pre-moistened towelettes, wet wipes, baby wipes and the like.

These and other features of the present subject matter will become readily apparent upon further review of the following specification.

### BRIEF DESCRIPTION OF THE DRAWINGS

FIG. 1 is a front view of a combination glove and antiseptic dispenser.

FIG. 2 is an exploded perspective view of the combination glove and antiseptic dispenser.

FIG. 3 is a perspective view of the combination glove and antiseptic dispenser.

Similar reference characters denote corresponding features consistently throughout the attached drawings.

### DETAILED DESCRIPTION OF THE PREFERRED EMBODIMENTS

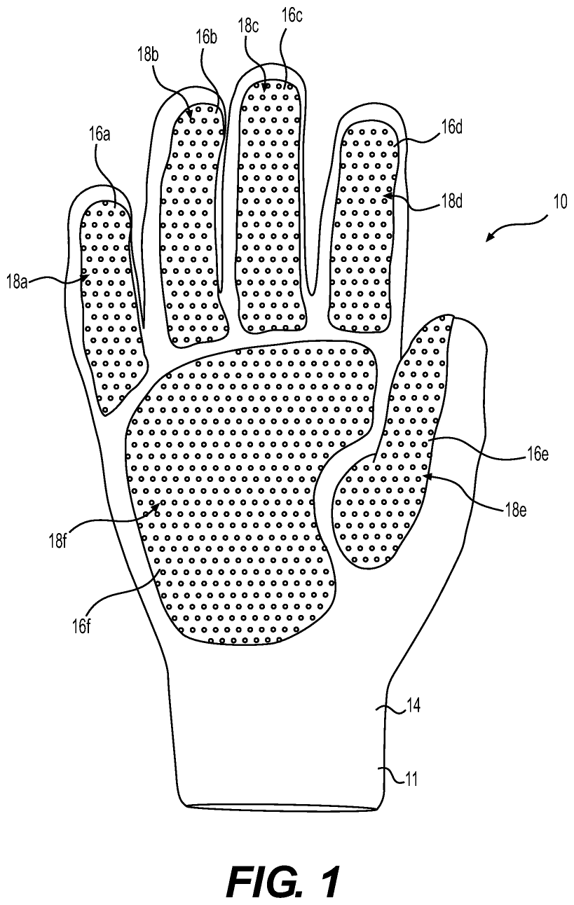

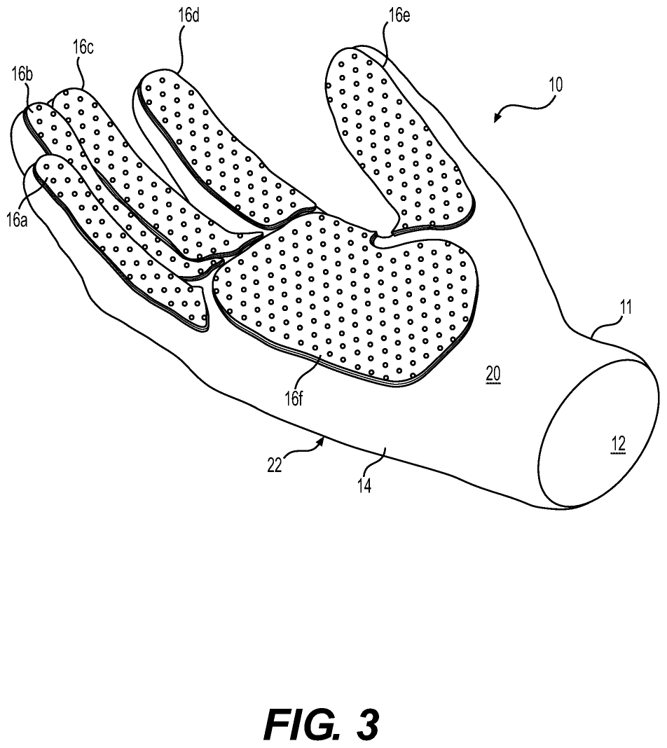

The combination glove and antiseptic dispenser 10 is a glove, for use in medical and laboratory environments, for example, to provide a fluid, microbial and viral impermeable barrier for the user's hand and, additionally, for dispensing an antiseptic liquid. As used herein, the term “antiseptic liquid” may be any suitable type of antiseptic agent, antimicrobial agent, antiviral agent and/or antibacterial agent. Non-limiting examples of such antiseptic liquids include chlorine dioxide solution, a suspension of silver nanoparticles, solutions of hydrocarbon sterilizing agents and the like. As shown in FIGS. 1-3, the combination glove and antiseptic dispenser 10 includes a glove 11, which is adapted for receiving the hand of the user and, as shown, is configured in a manner similar to a conventional nitrile rubber glove. The glove 11 has an inner surface 12 and an outer surface 14. When worn, the inner surface 12 contacts the skin of the user's hand, and the outer surface 14 is exposed to the environment. The glove 11 is formed from a first fluid-impermeable material.

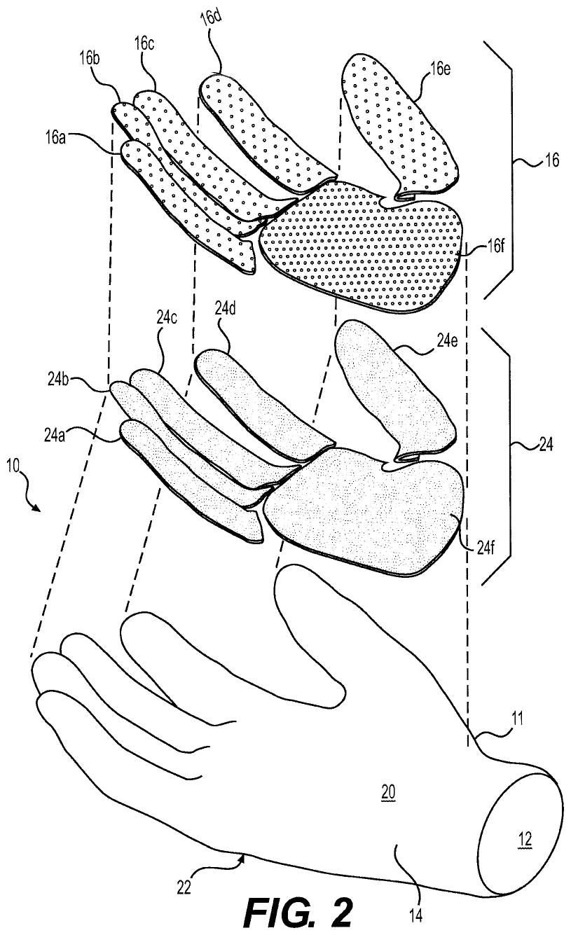

As best seen in FIG. 2, a dispensing layer 16 is provided. Dispensing layer 16 is formed from at least one sheet. In the non-limiting example of FIGS. 1-3, six such sheets are shown, with sheets 16a, 16b, 16c, 16d and 16e, respectively corresponding to the five fingers of the glove 11, and sheet 16f corresponding to the palm of the glove 11. Each of sheets 16a, 16b, 16c, 16d, 16e and 16f is made from a second fluid-impermeable material and has a plurality of pores 18a, 18b, 18c, 18d, 18e and 18f, respectively, formed therethrough. The first fluid-impermeable material of glove 11 may be the same as the second fluid-impermeable material of sheets 16a, 16b, 16c, 16d, 16e and 16f. A non-limiting example of such a material is nitrile rubber.

At least one absorbent layer 24 including an antiseptic liquid is sandwiched between the outer surface 14 of the glove 11 and the dispensing layer 16, such that the antiseptic liquid absorbed by the at least one absorbent layer 24 may be dispensed through the plurality of pores 18a, 18b, 18c, 18d, 18e and 18f. In the non-limiting example of FIG. 2, six such absorbent layers are shown, with absorbent layers 24a, 24b, 24c, 24d and 24e, respectively corresponding to the five fingers of the glove 11, and absorbent layer 24f corresponding to the palm of the glove 11. It should be understood that the at least one absorbent layer 24 may be formed from any suitable type of absorbent material, such as those commonly associated with pre-moistened towelettes, wet wipes, baby wipes and the like. In the above non-limiting examples, the at least one absorbent layer 24 and the at least one dispensing layer 16 are secured to the front face 20 of the glove 11 (i.e., the face covering the user's palm and which is opposed to the back face 22, which covers the back of the user's hand), however, it should be understood that the at least one absorbent layer 24 and the at least one dispensing layer 16 may be secured to any desired portion of the outer surface 14 of the glove 11.

The pores 18a, 18b, 18c, 18d, 18e and 18f formed through the second fluid-impermeable material may each have a diameter less than 1.3 mm (approximately 0.05 inches), and each preferably has a diameter less than 0.13 mm (approximately 0.005 inches). The diameter of the pores is configured to prevent unwanted leakage of the antiseptic liquid therethrough. In use, the act of grasping an object, pressing on a surface with the palm and fingers, squeezing the fist, rubbing two gloves together, etc. acts to drive the antiseptic liquid through pores 18a, 18b, 18c, 18d, 18e and 18f, thus disinfecting the desired object, surface, etc. or the gloves themselves. Following use, the glove 10 may be disposed of, similar to a conventional medical glove.

It should be understood that the dispensing layer 16 may be secured to outer surface 14 of glove 11 by any suitable type of attachment. Non-limiting examples include using adhesives, heat sealing and the like to secure the borders of dispensing layer 16 to outer surface 14, thus trapping the at least one absorbent layer 24 therebetween.

It is to be understood that the combination glove and antiseptic dispenser is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1. A combination glove and antiseptic dispenser, comprising:
a glove, the glove comprising five finger receptacles and a palm portion, the glove being adapted for receiving a hand of a user and having an inner surface and an outer surface, the glove being formed from a first fluid-impermeable material;
a dispensing layer comprising plurality of sheets formed from a second fluid-impermeable material, each of the plurality of sheets having a plurality of pores formed therethrough, each of the pores has a diameter less than 1.3 mm, wherein the plurality of sheets comprises six sheets respectively corresponding to the five finger receptacles and the palm portion of the glove; and
plurality of absorbent layers sandwiched between the outer surface of the glove and the plurality of sheets of the dispensing layer, each of the plurality of absorbent layers containing an antiseptic liquid adapted to be dispensed through a selected plurality of pores of each of the dispensing layers based on the pressure exerted by the user at that location, wherein the plurality absorbent layers comprises six absorbent layers respectively corresponding in size, configuration, and alignment with the corresponding six dispensing layers on the fingers and palm portion,
whereby movement of the user's hand in the glove dispenses an amount of the antiseptic liquid from the absorbent layer through the plurality of pores in the dispensing layer.

2. The combination glove and antiseptic dispenser as recited in claim 1, wherein the first fluid-impermeable material comprises the same material as the second fluid-impermeable material.

3. The combination glove and antiseptic dispenser as recited in claim 2, wherein the first fluid-impermeable material and the second fluid-impermeable material each comprise nitrile rubber.

4-8. (canceled)

9. The combination glove and antiseptic dispenser as recited in claim 1, wherein each of the pores has a diameter less than 0.13 mm.

10. A combination glove and antiseptic dispenser, consisting of:
a glove, the glove consisting of five finger receptacles and a palm portion, the glove being adapted for receiving a hand of a user and having an inner surface and an outer surface, the glove being formed from a first fluid-impermeable material;
a dispensing layer consisting of a plurality of sheets formed from a second fluid-impermeable material, each of the plurality of sheets having a plurality of pores formed therethrough, each of the pores has a diameter less than 1.3 mm, wherein the plurality of sheets consists of six sheets respectively corresponding to the five finger receptacles and the palm portion of the glove; and
plurality of absorbent layers sandwiched between the outer surface of the glove and the plurality of sheets of the dispensing layer, each of the plurality of absorbent layers containing an antiseptic liquid adapted to be dispensed through a selected plurality of pores of each of the absorbent layers based on the pressure exerted by the user at that location, wherein the plurality absorbent layers consists of six absorbent layers respectively corresponding in size, configuration, and alignment with the corresponding six dispensing layers on the fingers and palm portion,
whereby movement of the user's hand in the glove dispenses an amount of the antiseptic liquid from the absorbent layer through the plurality of pores in the dispensing layer.
